(19) United States
(12) Patent Application Publication
(10) Pub. No.: US 2009/0204210 A1
(43) Pub. Date: Aug. 13, 2009

(54) Accommodative Intraocular Lens

(76) Inventor: Joel Pynson, Toulouse (FR)

Correspondence Address: Bausch & Lomb Incorporated, One Bausch & Lomb Place, Rochester, NY, 14604-2701, US

(21) Appl. No.: 12/430,499

(22) Filed: Apr. 27, 2009

Related U.S. Application Data
(63) Continuation of application No. 11/284,381, filed on Nov. 21, 2005.

(51) Int. Cl. A61F 2/16

(52) U.S. Cl. 623/6.34

(57) ABSTRACT

An intraocular lens (IOL) comprising an apparatus capable of changing power in response to ciliary body movement. An IOL is provided that comprises a first optical power element, and a second optical power element. The second optical power element is mechanically coupled to the first optical power element, and at least one of the first optical power element and the second optical power element is mechanically coupled to at least one magnet, such that a magnetic field applied to the at least one magnet causes the first optical element and the second optical element to displace relative to one another. The optical power elements may be surfaces or lens, the magnetic medium may be liquid, gel or solid.

### CROSS-REFERENCE

This application is a continuation of application Ser. No. 11/284,381 filed Nov. 21, 2005, which is hereby incorporated by reference herein.

### FIELD OF INVENTION

The present invention relates to accommodative, intraocular lens systems, and more particularly to accommodative, intraocular lens systems capable of varying optical power in response to ciliary body and/or zonular movement.

### BACKGROUND OF THE INVENTION

There is seen in FIG. 1 a cross-sectional view of a human eye 10 having an anterior chamber 12 and a posterior chamber 14 separated by iris 30. Within the posterior chamber 14 is a capsular bag 16 which holds the eye's natural crystalline lens 17. Light enters the eye by passing through cornea 18 to the crystalline lens 17. The cornea and crystalline lens act together to direct and focus the light onto retina 20. The retina is connected to optic nerve 22 which transmits images received by the retina to the brain for interpretation.

In response to the sharpness of the image received by the retina, the brain contracts or relaxes ciliary muscle 26. In particular, to achieve near focus accommodation, the ciliary muscle is contracted thereby relaxing tension on zonules 27 which permits the capsular bag and lens 17 to become more rounded. To achieve far focus, the ciliary muscle is relaxed thereby increasing tension on zonules 27 which permits the capsular bag and lens 17 to become flatter. The ciliary muscle is disposed within the ciliary body 28, and upon contraction of the ciliary muscle, the ciliary body is caused to move.

In an eye where the natural crystalline lens has been damaged (e.g., clouded by cataracts), the natural lens is no longer able to properly focus and/or direct incoming light to the retina. As a result the images become blurred. A well known surgical technique to remedy this situation involves removal of the damaged crystalline lens and replacement with an artificial lens known as an intraocular lens (IOL), such as prior art IOL 24 seen in FIG. 2.

Conventional IOLs are typically fixed-focus lenses. Such lenses are usually selected to have a power such that the patient has a fixed focus for distance vision, and the patient requires spectacles or contact lens to permit near vision. In recent years extensive research has been carried out to develop accommodative IOLs (AIOLs) that permit the wearer to have accommodative vision.

Such AIOLs have included both single and dual lens systems that are located in the posterior chamber (e.g., in the capsular bag) and provide variable focal power in accordance with the pressure or tension exerted on the capsular bag 16 in accordance with contraction and relaxation of the ciliary muscle. However, to date, such systems have provided limited success. Although the exact reason for the limited success has not been identified, the unpredictable nature of the capsular bag and/or the zonules subsequent to surgery has contributed to the limited success. For example, post-surgical retraction and scarring have affected the performance of the bag.

Other conventional accommodative lenses have been proposed that include one or more electrically or piezolectrically-activated devices to effect changes in focal power of an AIOL. However, such lenses have tended to be complicated. For example, in some such devices, a source of electric power must be provided and numerous mechanical parts may be necessary.

### SUMMARY

Aspects of the present invention are directed to methods and apparatus of accommodation that provide accommodation at least partially independent of the zonules and/or independent of the mechanical properties of the capsular bag. According to aspects of the invention at least one magnet is coupled to the ciliary body and/or zonules and at least one magnet is provided on the IOL such that the lens focuses in response to movement of the ciliary body and/or zonules. It is to be appreciated that, in some embodiments, the use of one or more magnetic media may obviate the need for a source of electric power to achieve accommodation. It is to be further appreciated that the use of a magnetic medium to activate the lens may result in a reduced number of mechanical parts (e.g., gears) to achieve accommodation, thereby increasing reliability of the lens. The IOLs are sized and shaped to fit with a patient's eye; and in some embodiments may be sized and shaped to fit with a patient's capsular bag.

A first aspect of the invention is directed to an intraocular lens (IOL), comprising a first optical power element, a second optical power element coupled to the first optical power element, and at least one of the first optical power element and the second optical power element being mechanically coupled to at least one first magnetic medium, such that a magnetic field applied to the at least one first magnetic medium causes the IOL to change optical power.

The first optical power element may comprise a first surface of the IOL and the second optical power may comprise a second surface of the IOL. In some embodiments, at least one of the first surface and the second surface is flexible. In some embodiments, the first optical power element and the second optical power element are coupled together to form an enclosed space between the first optical power element and the second optical power element. The enclosed space may be filled with a gas or a fluid. The first magnetic medium may comprise a solid. The first magnetic medium may comprise a permanent magnet.

In some embodiments, the first optical power element comprises a first lens and the second optical power element comprises a second lens. In some embodiments, the first lens and the second lens are configured to translate without bending.

The first lens and the second lens may be coupled together by a hinge. The first lens may be coupled to the hinge by a first rigid element and the second lens may be coupled to the hinge by a second rigid element. The hinge may be a living hinge.

In some embodiments, the first magnetic medium is flowable. For example, the first magnetic medium may be comprised of a ferrofluid. The first optical power element and the second optical power element may be coupled together to form an enclosed space including a second medium, and the IOL may be configured such that, upon displacement of the first magnetic medium, the second medium is displaced in a manner to flex the first optical power element and the second optical power element. The first magnetic medium and the second medium may be separated by a movable barrier.

The IOL may comprise at least a first haptic in which the first magnetic medium is disposed. In some embodiments, the IOL comprises at least a second haptic in which a second magnetic medium is disposed. In some embodiments, the IOL comprises at least a third haptic in which a third magnetic medium is disposed. In some embodiments, the IOL comprises at least a fourth haptic in which a fourth magnetic medium is disposed.

The IOL may be in a combination with a ring sized and shaped to surround an eye, the ring maintaining at least a first magnet. In such embodiments, the IOL may further comprise a second magnetic medium mechanically coupled to the IOL, wherein the ring maintains a second magnet, the first magnet and the second magnet are disposed such that when the ring is placed around the IOL, the first magnetic medium is substantially opposite the first magnet and the second magnetic medium is substantially opposite the second magnet.

According to another aspect of the invention, an IOL is configured to change optical power in direct response to movement of at least one of the ciliary body and the zonules.

The IOL may comprise a first optical power element, and a second optical power element coupled to the first optical power element, and at least one of the first optical power element and the second optical power element being mechanically coupled to at least one first magnetic medium, such that a magnetic field applied to the at least one first magnetic medium causes the IOL to change optical power.

The first optical power element may be a first surface of the IOL and the second optical power is a second surface of the IOL. In such embodiments, the first optical power element and the second optical power element may be coupled together to form an enclosed space between the first optical power element and the second optical power element. The first magnetic medium may be a solid.

In some embodiments, the first optical power element comprises a first lens and the second optical power element comprises a second lens. The first magnetic medium may be flowable. The IOL may comprise at least a first haptic in which the first magnetic medium is disposed. In some embodiments, the IOL comprises at least a second haptic in which a second magnetic medium is disposed.

The IOL may be in a combination with a ring sized and shaped to surround an eye, and maintaining at least a first magnet. The IOL may further comprise a second magnetic, and the ring may maintain a second magnet; in such embodiments, the ring be sized and shaped such that when the ring is placed proximate the IOL, the first magnetic medium is substantially opposite the first magnet and the second magnetic medium is substantially opposite the second magnet.

The IOL may further comprise at least one magnetic medium configured and arranged such that a magnetic field applied to the at least one magnetic medium causes the IOL to change optical power. The IOL may be in a combination with at least one magnet shaped and sized to be attached to the ciliary body.

### BRIEF DESCRIPTION OF THE DRAWINGS

Illustrative, non-limiting embodiments of the present invention will be described by way of example with reference to the accompanying drawings, in which the same reference number is used to designate the same components in different figures, and in which:

FIG. 1 is a cross sectional side view of an eye including a natural, crystalline lens;

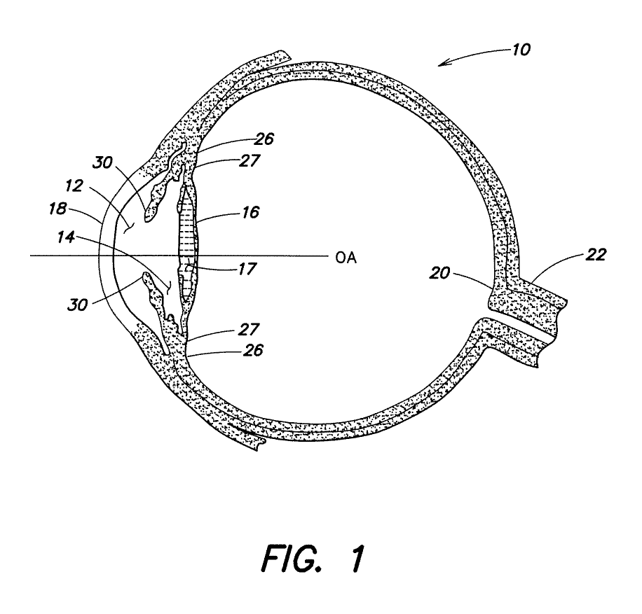

FIG. 2 is a cross sectional side view of an eye including an intraocular lens placed within the capsular bag;

FIGS. 3A and 3B are cross sectional side views of an example of an embodiment of a lens according to aspects of the present invention;

FIGS. 4A and 4B are cross sectional side views of a second embodiment of a lens according to aspects of the present invention;

FIG. 4C is a perspective view of an example of the second embodiment of a lens;

FIG. 4D is a perspective view of another example of the second embodiment of a lens;

FIGS. 5A and 5B are cross sectional side views of another embodiment of a lens according to aspects of the present invention; and

FIGS. 5C and 5D are perspective views of an example of an embodiment of a lens according to the embodiment illustrated in FIGS. 5A and 5B.

### DETAILED DESCRIPTION

Aspects of the present invention are directed to an intraocular lens (IOL) comprising an apparatus capable of changing power in response to ciliary body movement and/or direct response to zonule movement. An advantage of embodiments of such IOLs capable of changing power in direct response to zonule movement is that accommodation can occur despite a reduced ability or non-ability of the capsular bag to move in response to movement of the ciliary body. An advantage of embodiments of IOLs capable of changing power in direct response to ciliary body movement is that accommodation can occur despite a reduced ability or non-ability of the zonules and/or capsular bag to move in response to movement of the ciliary body.

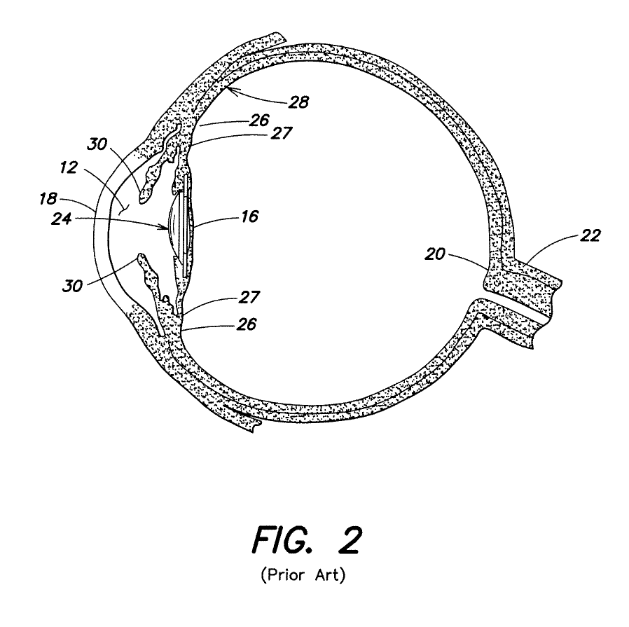

According to some aspects of the invention, an IOL is provided that comprises a first optical power element, and a second optical power element. According to such aspects, the second optical power element is mechanically coupled to the first optical power element, and at least one of the first optical power element and the second optical power element is mechanically coupled to at least one magnet, such that a magnetic field applied to the at least one magnet causes the first optical element and the second optical element to displace relative to one another. An advantage of embodiments of such systems is that accommodation of the lens can occur in response to a magnetic field thereby, in some embodiments, obviating the need for a power source and/or gearing to achieve accommodation. Accordingly, the likelihood of failure of such a system may be reduced. The IOL may be inserted into the capsular bag as illustrated in FIG. 2 or other suitable location.

It is to be appreciated that the phrase “in response to movement of the ciliary body” includes embodiments where accommodation is achieved in direct response to movement of the ciliary body, as well as, embodiments where accommodation is achieved in indirect response to movement of the ciliary body. Accommodation “in direct response to movement of the ciliary body” means that the amount of accommodation achieved is directly determined at least in part by the movement of the ciliary body without requiring the force generated by the ciliary body to be applied using the zonules or the capsular bag (e.g., accommodation of an IOL may be achieved in direct response to movement of a ciliary body using a magnetic field, the magnetic field being controllable by movement of the ciliary body so as to operate on a magnet coupled to the IOL as described herein). It is to be appreciated that accommodation “in direct response to movement of the ciliary body” may be achieved with the zonules and/or capsular bag intact, and the zonules and/or capsular bag may, in part, impact the amount of accommodation achieved. For example, accommodation “in direct response to movement of the ciliary body” may be achieved by attaching a first magnetic medium to the ciliary body and attaching a second magnetic medium to an IOL, as described herein, whereby movement of the ciliary body results in accommodation of the IOL.

Accommodation “in direct response to movement of at least one of the ciliary body and the zonules” means that the amount of accommodation achieved is determined by the movement of the ciliary body and/or zonules without requiring force to be applied using the capsular bag. It is to be appreciated that accommodation “in direct response to movement of at least one of the ciliary body and capsular bag” may be achieved with the capsular bag intact, and the capsular bag may, in part, impact the amount of accommodation achieved.

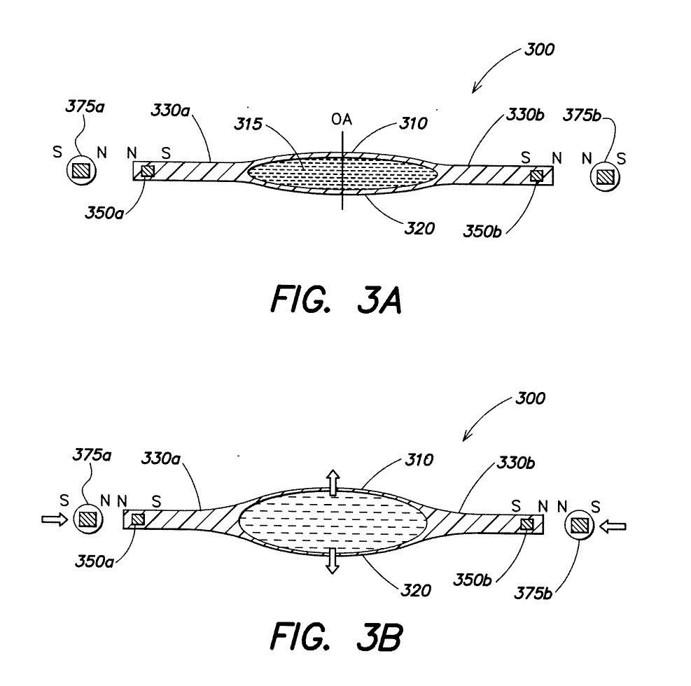

FIGS. 3A and 3B are cross sectional side views of an example of an embodiment of an intraocular lens (IOL) 300 according to aspects of the present invention. IOL 300 includes a first optical power element, constituting a first surface 310 of a lens comprising IOL 300, and a second optical power element, constituting a second surface 320 of the lens comprising IOL 300.

First surface 310 and second surface 320 are mechanically coupled to first magnet 350a and second magnet 350b, such that a magnetic field applied to first and second magnets 350a and 350b by first ciliary magnet 375a and second ciliary magnet 375b, respectively, causes the first surface 310 to displace relative to the second surface 320. In particular, magnet 350a and ciliary magnets 375a are arranged such that their common poles are facing one another (e.g., as illustrated, their N poles face one another) and are therefore repulsive of one another. Similarly magnet 350b and ciliary magnets 375b are arranged such that their north poles (N) are facing one another. It is to be appreciated that by displacing the first surface relative to the second surface, the power of IOL 300 changed. As ciliary magnets 375a and 375b approach magnets 350a and 350b, respectively, haptics 330a and 330b are pushed toward optical axis OA causing first surface 310 and second surface 320 to be increasingly separated from one another. It is to be appreciated that haptics 330a and 330b may be selected to have dimension so as to contact the capsular bag and thereby center IOL 300 within a patient's capsular bag.

As illustrated in FIG. 3A, when the ciliary muscle (not shown) is relaxed, the repulsive force between magnets 350a and 375a, and magnets 350b and 375b causes IOL 300 to reach an equilibrium with a determined amount of flexure of surfaces 310 and 320. The power provided by IOL 300 as determined by the shape and location of surfaces 310 and 320 is based on the magnetic properties of the magnets 350 and 375 and mechanical properties of IOL 300. As illustrated in FIG. 3B, upon contraction of the ciliary muscle, the ciliary magnets 375 move closer to a corresponding one of magnets 350, and as a result, surfaces 310 and 320 separate from one another, and the curvatures of surfaces 310 and 320 become greater such that the power of IOL 300 is increased. It is to be appreciated that the surfaces 310 and 320 may be predisposed to separation from one another upon contraction due to a curved shape of surfaces 310 and 320 (e.g., a convex shape). Separation will typically be most pronounced along axis OA. It is to be appreciated that intraocular lens 300 is capable of changing power in direct response to ciliary body movement.

Surfaces 310 and 320 are comprised of materials capable of flexing a sufficient amount to achieve a suitable change in power of IOL 300. In the illustrated embodiment, the materials are selected to have a suitable transparency to visible light such that an image of adequate brightness can be formed on a patient's retina.

In some embodiments, the magnets comprise a suitable solid, permanent magnet. For example, any of the magnets can comprise one or more of the following metallic or ceramic, magnetic materials: Neodynium Iron Boron, Samarium Cobalt or Aluminum Nickel Cobalt. These materials may be suitably shaped. For example, the magnets may be configured as balls, blocks, wires or rods. The magnets may be sheathed in a biologically inert material (e.g., silicone) as may be desirable.

First optical power element 310 and second optical power element 320 may be mechanically coupled together by any suitable technique. The first and second surfaces define an interior space 315. In some embodiments, first and second surfaces are coupled together such that interior space 315 is completely enclosed. However, embodiments of the invention are not limited to such an enclosure, and one or more openings may be present. For example, one or more openings may be formed around the periphery of IOL 300.

In embodiments in which interior space 315 is completely enclosed, the interior space may be filled by a gaseous medium (e.g. air) or a fluid medium (e.g., a liquid or a gel). An advantage of a fluid medium is that it may have a higher index of refraction than a gas such as air. In embodiments in which the surfaces do not enclose the interior space, aqueous fluid that is present in the anterior chamber of the eye would typically be present in the interior space when the lens is implanted in the eye.

Although the magnets 375a and 375b are designated herein as ciliary magnets, this designation is given merely as an example. The magnets so designated may be attached to one or more of the ciliary body and the zonules. Where the magnets 375 are coupled is determined at least in part by which of these locations is capable of movement in response to a natural nerve stimulus from the brain that indicates that focusing of the lens is to occur. A suitable capability of movement of any of the above locations, which determines at least in part a suitable location of magnets 375, will be determined by a patient's physiological condition. As one of ordinary skill in the art would understand, the ciliary body receives a nerve impulse and reacts to the impulse. By contrast, the zonules respond to the ciliary body movement and respond only indirectly to a nerve impulse. Accordingly, there is typically a greater likelihood that capability of movement will be present in the ciliary body than the zonules. Any suitable technique of attachment to a selected location may be used, for example, surgical implantation into the location, adhering onto the location or mechanical fastening to the location.

In some embodiments, it is desirable that a lens provide 5 to 6 diopters of accommodation upon movement of the lens in response to the movement of magnets 375. Accordingly, after determining the amount of movement which an identified location is capable of and determining a desirable amount of accommodation, an IOL may be designed or selected. For example, the lens should be selected to have a suitable magnet strength, suitable mechanical characteristics (e.g., surface flexibility), and suitable lens surface curvatures.

Although two magnets 350a and 350b are illustrated, any suitable number of magnets (e.g., 1, 3 or 4) may be included. For each magnet 350 that is included, an equal number of ciliary magnets may be included, each arranged to be repulsive, as described above; however, the number of magnets 350 and magnets 375 may be different than one another. It is to be appreciated that a first number of magnets may be implanted initially and further magnets may be later added or removed as determined to be medically desirable to achieve a suitable amount of accommodation (e.g., to achieve 5-6 diopters of accommodation).

In some embodiments, each magnet 350 is included in a corresponding haptic 330a and 330b. However, a single haptic may extend around a circumferential portion of IOL 300 so as to include more than one magnet 350, each arranged to interact with one or more ciliary magnets 375. In some embodiments a single haptic may extend completely around the circumference of IOL 300.

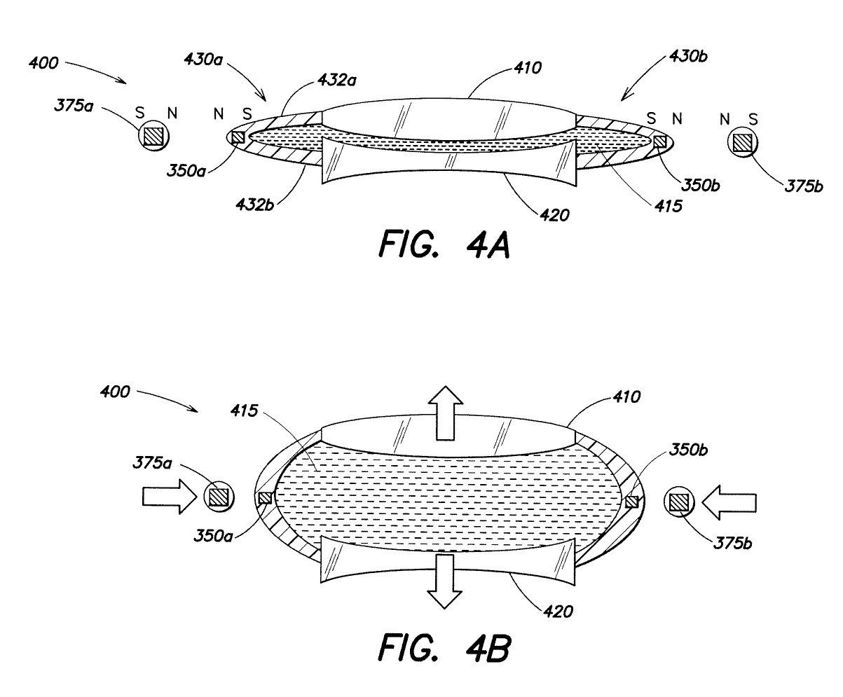

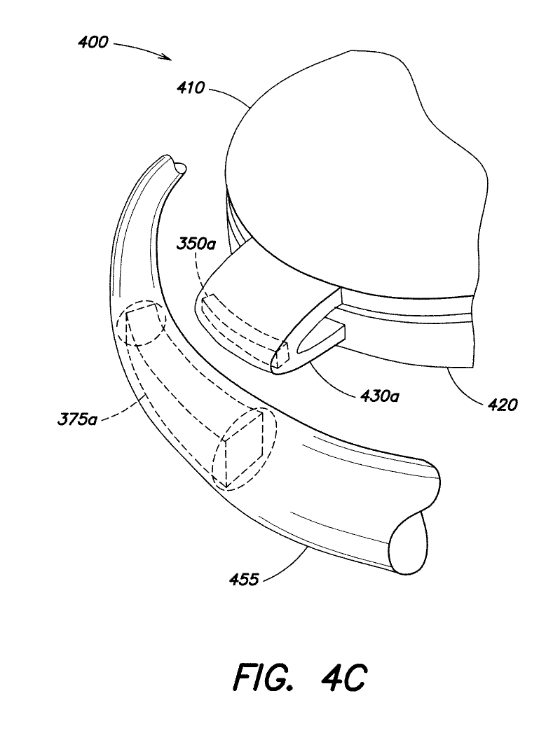

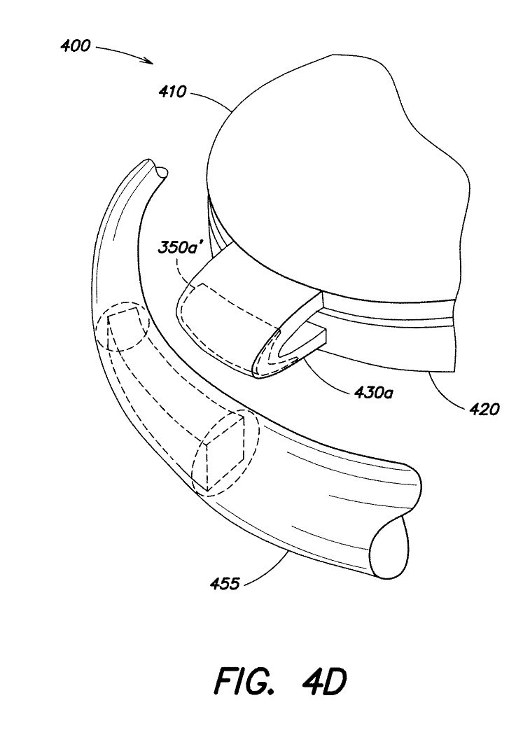

FIGS. 4A and 4B are cross sectional side views of another embodiment of an IOL 400 according to aspects of the present invention. IOL 400 includes a first optical power element, constituting a first lens 410 of the IOL 400, and a second optical power element, constituting a second lens 420 of IOL 300. In some embodiments, first lens 410 and second lens 420 may be connected together by a structure 430a. However, first lens 410 and second lens 420 may be coupled together by any suitable structure that permits first lens 410 and second lens 420 to translate relative to one another such that a magnetic field applied to magnets 350 causes IOL 400 to change optical power. The structure may include any include a suitable synthetic material and/or a patient's own biological material.

As with the apparatus described above with reference to FIGS. 3A and 3B, first optical power element (i.e., lens 410) and second optical power element (i.e., lens 420) are mechanically coupled to first magnet 350a and second magnet 350b, such that a magnetic field applied to first and second magnets 350a and 350b by first ciliary magnet 375a and second ciliary magnet 375b, respectively, causes the first lens 410 to displace relative to the second lens 420. In particular, as with the device in FIGS. 3A and 3B, magnet 350a and ciliary magnets 375a are arranged such that their common poles are facing one another (e.g., as illustrated their N poles) and are therefore repulsive of one another. Similarly, magnet 350b and ciliary magnets 375b are arranged such that their north poles (N) are facing one another. It is to be appreciated that displacing the first lens 410 relative to the second lens 420 causes the power of IOL 400 to be changed. Any suitable number of magnets 350 and 375 may be used.

Structures 430a and 430b could comprise any suitable apparatus that causes first lens 410 and second lens 420 to translate upon application of magnetic force to magnets 350a and/or 350b. For example, structure 430a and 430b may comprise a flexible material 430 that is flexible enough to bend in the region of magnet 350a in response to a magnetic field applied to magnets 350a and 350b, yet rigid enough to move lenses 410 and 420 apart upon application of the magnetic force.

Alternatively, structure 430a and 430b could comprise rigid segments 432a and 432b that pivot about magnet 350a with no substantial flexing of either rigid segment. For example, a magnet 350 may be connected to a hinge such that rigid segments 432a and 432b pivot about the hinge. It is to be appreciated that the hinge could be constructed by forming a suitable region of thinness (i.e., a living hinge) in structure 430a at magnet 350a such that the region would permit pivoting of rigid segments 432a and 432b about magnet 350a in response to magnetic force applied to magnet 350a.

As illustrated in FIG. 4A, when the ciliary muscle (not shown) is relaxed, the repulsion reaches an equilibrium based on the magnetic properties of the magnets 350 and 375 and mechanical properties of IOL 400. For example, as illustrated in FIG. 4B, upon contraction of the ciliary muscle the ciliary magnets 375 move closer to magnets 350 and lenses 410 and 420 separate. Accordingly, intraocular lens 400 is capable of changing power in response to ciliary body movement. It is to be appreciated that some embodiments of a lens system including a structure 430a, 430b are capable of causing a power change by only translation of the first power element relative to the second power element (e.g., no bending of the surfaces of lens 410 or 420 is provided to change the power of IOL 400).

IOL 400 may comprise any suitable combination of lenses 410, 420 capable of providing a change in power of IOL 400 upon translation of lenses 410 and 420 relative to one another. As illustrated in the FIG. 4A, lens 410 may be selected to be a positive lens and lens 420 is selected to be a negative lens, such that when lens 410 and 420 move apart from one another, the focal power of IOL 400 is increased. IOL 400 may comprise more than two lenses.

The first and second lenses 410 and 420 and structure 430 define an interior space 415. In some embodiments, the first and second lenses are coupled together such that interior space 415 is completely enclosed. However, embodiments of the invention are not limited to such an enclosure, and one or more openings may be formed around the periphery of the IOL 400. In embodiments in which interior space 415 is completely enclosed, the interior space may be filled a gaseous medium or a fluid medium.

FIG. 4C is a perspective view of an example of a lens according to the second embodiment. Magnet 375a is disposed within a ring of material 455 that surrounds IOL 400. For example, ring of material 455 may be attached to the ciliary body by an adhesive, a mechanical fastener, surgically or other suitable technique. For example, ring of material 455 may be attached to the pars plicatura or the zonules. Magnet 375a is disposed opposite magnet 350a such that, as ring 455 is displaced in response ciliary muscle contraction and relaxation, structure 430a operates to translate lens 410 relative to lens 420.

FIG. 4D is a perspective view of another example of lens according to the second embodiment of a lens. The exemplary lens in FIG. 4D is similar to the lens in FIG. 4C except magnet 430a is wedge-shaped so as to substantially conform to haptic 430a.

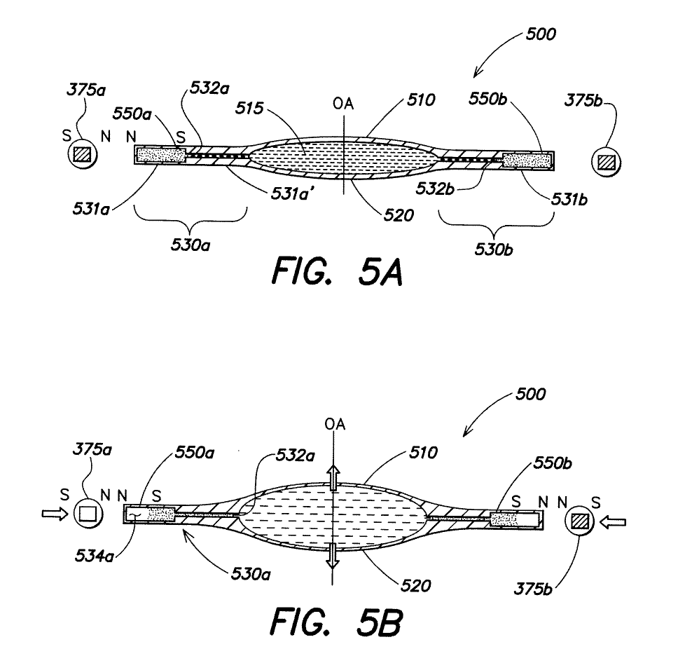

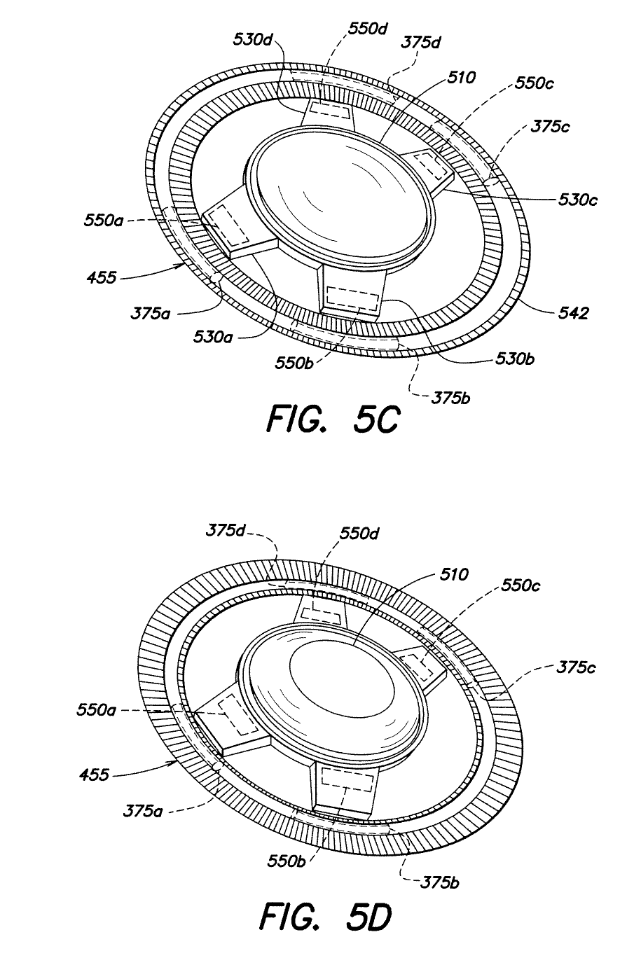

FIGS. 5A and 5B are cross sectional side views of another embodiment of an IOL 500 according to aspects of the present invention. Similar to the IOL illustrated in FIGS. 3A and 3B, IOL 500 includes a first optical power element, constituting a first surface 510 of a lens comprising the IOL 500, and a second optical power element, constituting a second surface 520 of a lens comprising IOL 500.

First surface 510 and second surface 520 are mechanically coupled to first magnet medium 550a and second magnet medium 550b, such that a magnetic field applied to first and second magnets 550a and 550b by first ciliary magnet 375a and second ciliary magnet 375b, respectively, causes the first surface 510 to displace relative to the second surface 520. In the embodiment illustrated in FIGS. 5A and 5B, the magnetic media are flowable magnetic medium. For example, magnetic media 550a and 550b may be magneto-rheological fluid such as a ferrofluid containing nanograins. First surface 510 and second surface 520 may be mechanically coupled together such that interior space 515 is completely enclosed. Interior space may be filled a gaseous medium (e.g. air) or a fluid medium (e.g., a liquid or a gel).

Magnetic media 550a and 550b are preferably maintained separately of the medium in the interior space such that magnetic medium 550a is maintained in a portion 531a of haptic 530a and a portion of the medium in the interior space 515 is disposed in a portion 531a′ of haptic 530a. Similarly a magnetic medium 550b is maintained in a portion 531b of haptic 530b. For example, movable barriers 532a and 532b may be disposed in haptics 531a and 530b between magnetic media 550a and 550b such that the magnetic media do not mix with the fluid or gas in interior space 515. In some embodiments, a surfactant may be provided to the magnetic media to prevent conglomeration.

As one of ordinary skill in the art would understand, as illustrated in FIG. 5B, when a ferrofluid is subjected to a magnetic field, particles of the ferrofluid move in the direction of the magnetic flow, which results in movement of the fluid itself. Accordingly, a void 534a may be formed in haptic 530a as the particles of the ferrofluid move radially inward, and medium in interior space 515 is displaced such that surfaces 510 and 520 are made to be more convexly curved.

As illustrated in FIG. 5A, when the ciliary muscle (not shown) is relaxed, the displacement of the medium in the interior space 515 reaches an equilibrium based on the magnetic properties of the magnetic media 550a and 550b and magnets 375a and 375b, and mechanical properties of the IOL (e.g., the flexibility of surfaces 510 and 520). As illustrated in FIG. 5B, upon contraction of the ciliary muscle, ciliary magnets 375a and 375b move closer to magnetic media 550a and 550b, respectively, thereby causing first surface 510 and second surface 520 flex and separate from one another. It is to be appreciated that the separation is most pronounced along axis OA such that the curvatures of surfaces 510 and 520 become greater and the power of IOL 500 is increased. Accordingly, intraocular lens system 500 is capable of changing power in response to ciliary body movement.

Although two haptics are illustrated, each having magnetic media 550a and 550b disposed therein are illustrated, any suitable number of haptic including flowable magnetic media (e.g. 1, 3 or 4) may be included.

FIGS. 5C and 5D are perspective views of an example of an embodiment of a lens according to the embodiment illustrated in FIGS. 5A and 5B, in which the lens has four haptics 530a-530d. In the illustrated embodiment, ring 455 is attached to zonules 542. In FIG. 5C the ciliary muscle is relaxed as described with reference to FIG. 5A above, and ring 455 including ciliary magnets 375a-375d is uncompressed. Accordingly, magnetic media 550a-550d are disposed in locations in the radially outermost portions of the haptics 530a-530d; and surfaces 510 and 520 have relatively small curvatures.

In FIG. 5D, the ciliary muscle is contracted as described with reference to FIG. 5B above, and ring 455 including ciliary magnets 375a-375d is compressed radially inward by the ciliary body. Accordingly, magnetic media 550a-550d are disposed in the radially innermost portions of the haptics 530a-530d; and as a result, surfaces 510 and 520 are more curved than in FIG. 5C. It is to be appreciated that although surfaces 510 and 520 were described as both being flexible, they may have different flexibilities. In some embodiments, one of surfaces 510 and 520 may be rigid and only the other of surfaces 510 and 520 will attain greater curvature in response to ciliary movement.

Having thus described the inventive concepts and a number of exemplary embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

What is claimed is:

1. An intraocular lens (IOL), comprising:
a first optical power element comprising a first lens;
a second optical power element comprising a second lens element, the second power element coupled to the first optical power element; and
at least one haptic connected to the first optical power element and the second optical power element,
the first optical power element, the second optical element and the at least one haptic defining an interior space, the interior space having one or more openings to permit, when the lens is located in an eye, aqueous fluid of the eye to be present therein, and
at least one of the first optical power element and the second optical power element being mechanically coupled to at least one first magnetic medium, the lens being adapted to be inserted into a capsular bag of the eye, and the magnetic medium being disposed in the at least one haptic and arranged such that a magnetic field applied to the at least one first magnetic medium causes the IOL to change optical power.

2. The IOL in claim 1, wherein the first magnetic medium is solid.

3. The IOL in claim 2, wherein the first magnetic medium comprises a permanent magnet.

4. The IOL in claim 1, wherein the first lens and the second lens are configured to translate without bending.

5. The IOL in claim 4, wherein the first lens and the second lens are coupled together by a hinge.

6. The IOL in claim 5, wherein the first lens is coupled to the hinge by a first rigid element and the second lens is coupled to the hinge by a second rigid element.

7. The IOL in claim 6, wherein the hinge is a living hinge.

8. The IOL in claim 1, wherein the IOL comprises at least a second haptic in which a second magnetic medium is disposed.

9. The IOL in claim 8, wherein the IOL comprises at least a third haptic in which a third magnetic medium is disposed.

10. The IOL in claim 9, wherein the IOL comprises at least a fourth haptic in which a fourth magnetic medium is disposed.

11. The IOL of claim 1, in a combination with a ring shaped to surround the IOL and to be attached to a ciliary body of an eye, the ring maintaining at least a first magnet.

12. The combination of claim 11, further comprising a second magnetic medium mechanically coupled to the IOL, wherein the ring maintains a second magnet, the first magnet and the second magnet being disposed such that when the ring is placed around the IOL, the first magnetic medium is substantially opposite the first magnet and the second magnetic medium is substantially opposite the second magnet.
